(19) United States
(12) Patent Application Publication
(10) Pub. No.: US 2004/0176792 A1
(43) Pub. Date: Sep. 9, 2004

(54) Device and method for advancing a wire

(76) Inventor: David Rott, Modi ' in (IL)

Correspondence Address: ROBERT L STONE PC, 13 MEADOWLARK LN, EAST BRUNSWICK, NJ, 08816, US

(21) Appl. No.: 10/454,992

(22) Filed: Jun. 5, 2003

(30) Foreign Application Priority Data
Mar. 9, 2003 (IL) 154813

(51) Int. Cl. A61M 29/00

(52) U.S. Cl. 606/194

(57) ABSTRACT

A device for advancing a wire intravascularly including a wire advancement device (WAD) adapted to hold a wire to be advanced, while the WAD is advancing wire-over-wire over a guidewire already in place and a method for advancing a wire including holding the wire in a wire advancing device, and advancing the wire advancing device by wire-over-wire advancement over a guidewire already in place.

### FIELD OF THE INVENTION

The present invention relates to devices and procedures for introducing various medical instruments through the blood vessels of a patient, in general and, in particular, to a device and method for advancing a guidewire during intravascular procedures.

### BACKGROUND OF THE INVENTION

The Percutaneaus Coronary Intervention (PCI) procedure, and other procedures in interventional cardiology and interventional radiology requiring transfer of medical instruments through the blood vessels of a patient, involves transfer of a steerable guidewire to a target area inside the patient's body. In PCI procedures, the steerable guidewire must be transferred beyond a target coronary lesion. Once the guidewire is in place, it is possible to transfer various instruments over the wire, such as a PTCA dilatation catheter, a balloon expandable stent system, or other device.

Frequently, difficulties arise in advancing the guidewire into the target vessel, or across the lesion. These difficulties mandate the replacement (exchange) of the originally chosen guidewire by a more suitable wire (e.g., a stiffer guidewire or guidewire with a different curve).

One option for replacing the guidewire is the so-called “over-the-wire PTCA dilatation catheter”. This catheter includes a long guidewire receiving lumen, extending from the front end to the back end of the dilatation catheter. This catheter allows guidewire exchange without losing the advantage from having advanced the original wire a significant distance towards the target lesion. It also allows verification of guidewire position (in the vessel true lumen instead of in a false lumen) by contrast injection through the wire receiving lumen. The over-the-wire PTCA dilatation catheter suffers, however from the serious disadvantage of being cumbersome, mandating a special operating table, and the presence of two operators in order to manipulate a long (300 centimeters) guidewire.

This limitation was overcome when the so-called rapid-exchange technique was introduced. This technique utilizes a PTCA dilatation catheter having a short guidewire receiving barrel extending from a guidewire port at the distal end of the PTCA dilatation catheter to another port located about 25 cm proximally on the catheter side. Since only 25 cm of the 140 centimeters length of the PTCA dilatation catheter are over the wire, a shorter guidewire of 175 centimeters can be used. This allows rapid and more convenient dilatation catheters exchange by a single operator.

Using the rapid-exchange system, guidewire replacement requires complete withdrawal of the original guidewire and introduction of the new guide wire to the target vessel and then beyond the target coronary lesion. If difficulties arise in advancing the original guidewire in the vessel to reach the target lesion, it is likely that similar difficulties will arise when using the new guidewire. In any event, this situation is likely to lengthen the procedure, increase the danger of internal vessel injury from the guidewire, itself, as well as cause frustration for the operator. With the current rapid-exchange system, the operator does not get any advantage from having advanced the original wire a significant distance towards the target lesion, but must start from scratch with a new wire. Thus, the rapid-exchange system does not share the major advantages of the over-the-wire PTCA dilatation catheter system (e.g. it does not allow either convenient guidewire exchange, or verification of wire position by contrast injection).

U.S. Pat. No. 5,135,535 to Kramer suggests a solution for this problem. Kramer discloses an intravascular catheter system including a catheter comprising an elongated body having an inflation lumen for directing inflation fluid to the interior of an inflatable balloon on the catheter body, and a guidewire-receiving inner lumen extending along the length of the catheter. This catheter design permits the catheter to be used as an over-the-wire type PTCA dilatation catheter to advance to the location of the guidewire. If the guidewire must be replaced, the catheter remains in position while the original guidewire is extracted from the guidewire-receiving inner lumen. A new guidewire can now be introduced into the guidewire-receiving inner lumen until it reaches the distal end of the catheter, and continues to be advanced to the desired target. The catheter with balloon can now be transferred over the new wire to the target.

This system suffers from several disadvantages. First, when replacing a wire, the patented method requires the use of a long (300 centimeters) replacement guidewire, as used in the “over the wire” technique. Second, a special, complicated, dedicated PTCA dilatation catheter is required in order to perform the patented invention, so that conventional PTCA catheters cannot be employed.

Accordingly, there is a long felt need for a device to permit an operator, using the rapid-exchange system, to take advantage of the distance an original guidewire is advanced before replacement, and it would be very desirable if the device could be utilized with a shorter guidewire of 175 centimeters, as well as with conventional PTCA catheters and stents in use at present. It would be particularly desirable if the device would permit verification of wire position by contrast injection.

### SUMMARY OF THE INVENTION

The present invention provides a simple device for permitting replacement of a guidewire while taking advantage of the distance traveled successfully by the original guidewire.

There is thus provided, is accordance with the present invention, a device for advancing a wire intravascularly, the device including a wire advancement device (WAD) adapted to hold a wire to be advanced, while the WAD is advancing wire-over-wire over a guidewire already in place.

There is also provided, according to the invention, a device for advancing a wire, the device including a flexible rod defining two barrels along at least a portion of its length, one barrel being arranged to hold and advance a wire, and the second barrel being arranged for wire-over-wire advancement over a guidewire already in place.

According to one embodiment of the invention, device includes a flexible rod defining two elongate barrels, and an inflatable balloon lining an interior surface of each barrel, the balloons, in an inflated state, being adapted to securely hold a wire and, in a deflated state, permitting free movement along a wire, the balloons being coupled, via inflation tubes, to an inflation device.

According to an alternative embodiment of the invention, the WAD further includes a third barrel (or lumen), extending from the proximal end to the distal end of the WAD, for injection of a liquid through the WAD, particularly a contrast dye to allow verification of wire position.

There is also provided, in accordance with the present invention, a method for advancing a wire, the method including holding the wire in a wire advancing device, and advancing the wire advancing device by wire-over-wire advancement over a guidewire already in place.

According to one embodiment of the invention, the step of holding includes threading the wire through a first barrel in a flexible rod having two barrels, the first barrel being arranged to hold the wire, and threading an end of the guidewire through the second barrel, the second barrel being arranged for wire-over-wire advancement over the guidewire.

Preferably, the step of threading includes threading said wire through a side port in said first barrel, and through and out a distal end of said first barrel.

According to one embodiment, the method includes threading the wire through a first barrel of a wire advancement device (WAD), the first barrel being lined with a first inflatable balloon, inflating the first balloon so as to hold the wire, threading an outer end of the guidewire through a second barrel of the WAD, the second barrel being lined with a deflated second inflatable balloon, and advancing the WAD over the guidewire.

Further according to one embodiment, the method further includes deflating the first inflated balloon, so as to release the wire, inflating the second inflatable balloon so as to hold the guidewire, and withdrawing the WAD and guidewire over the wire According to an alternative embodiment of the invention, the method further includes injecting a contrast material through a third barrel (or lumen), extending from the proximal end to the distal end of the WAD to permit verification of the wire position

### BRIEF DESCRIPTION OF THE DRAWINGS

The present invention will be further understood and appreciated from the following detailed description taken in conjunction with the drawings in which:

FIG. 1A shows a device for intravascular advancement of a guidewire constructed and operative in accordance with one embodiment of the present invention;

FIGS. 1B and 1C are sectional illustrations through line A-A and B-B, respectively, in FIG. 1A;

FIGS. 2A, 2B, 2C, 2D and 2E are a schematic illustration of the method of operation of the device of FIG. 1;

FIG. 3A shows a device for intravascular advancement of a guidewire constructed and operative in accordance with another embodiment of the present invention;

FIGS. 3B and 3C are sectional illustrations through line A-A and B-B, respectively, in FIG. 3A;

FIG. 4a is a plan view of a device for intravascular advancement of a guidewire constructed and operative in accordance with a further embodiment of the present invention; and

FIGS. 4B and 4C are sectional illustrations through line A-A and B-B, respectively, in FIG. 4A.

### DETAILED DESCRIPTION OF THE INVENTION

The present invention relates to a new rapid exchange device, a Wire Advancement Device (WAD), for guidewire exchange during PCI and other similar procedures, or for advancing other medical carrier wires or special wires, particularly in interventional cardiology and interventional radiology. The WAD includes a flexible rod having a pair of barrels along a portion of its length, one barrel of which is arranged to hold and transfer a replacement or medical carrier wire, and the other barrel arranged to advance over a guidewire already in place, i.e., wire-over-wire transfer. According to one embodiment of the invention, the WAD further includes a third barrel (or lumen), extending from the distal end to the proximal end of the WAD, for injection of a liquid through the WAD, particularly a contrast dye to allow verification of wire position.

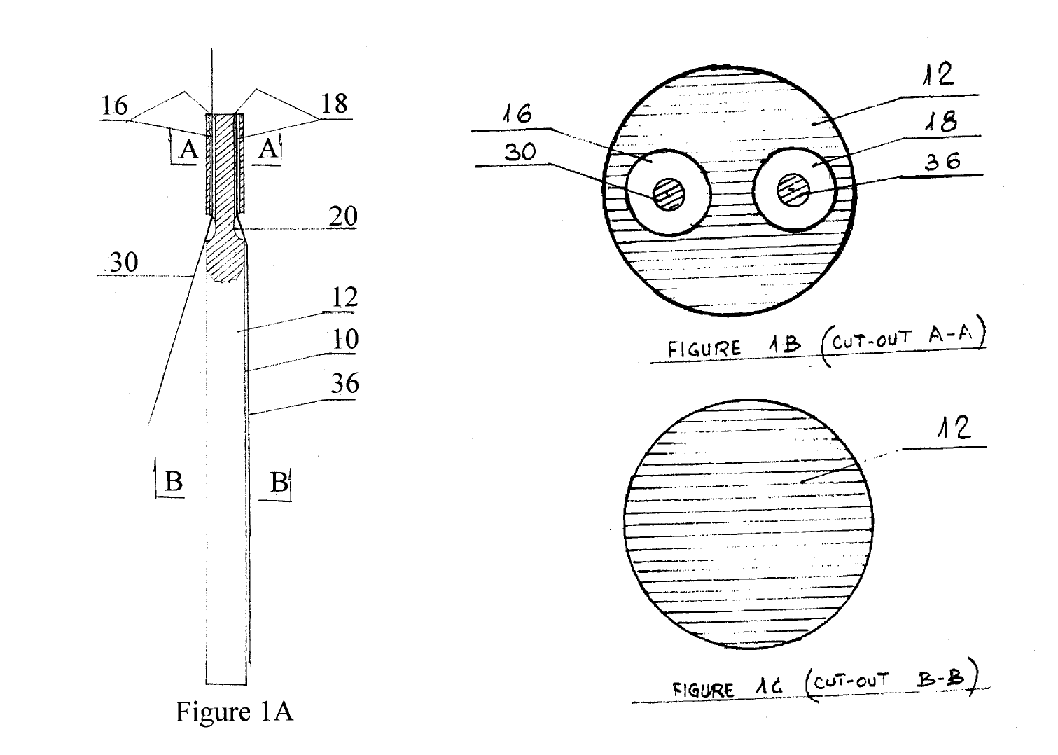

Referring now to FIG. 1A, there is shown a partially cut-away plan view of a wire advancement device (WAD) 10 for intravascular advancement of a wire, such as a guide wire, or other medical wire, constructed and operative in accordance with one embodiment of the present invention. WAD 10 adapted to hold a wire to be advanced, while the WAD is advancing wire-over-wire over a guidewire already in place. It was be appreciated by those skilled in the art that WAD 10 can be formed from any materials found useful in intravascular catheters.

Device 10 includes a flexible rod 12 (see FIG. 1C) with two longitudinal barrels 16 and 18 (seen sectional view in FIG. 1B). WAD 10 should be of low profile, its size being determined according to the diameter of the vessels through which it must pass. According to a preferred embodiment of the invention, WAD 10 is 0.9 millimeters (2.7 French), or less, in diameter and about 140 centimeters in length. Most preferably, the front 25 centimeters of WAD 10 define a pair of parallel barrels, extending from the distal end of WAD 10 and out the sides of WAD 10, as best seen in FIG. 1A.

According to one embodiment of the invention, WAD 10 further includes a side port 20 near the rear of each barrel, through which the guidewire extends, as known in conventional over-the-wire PCI instruments.

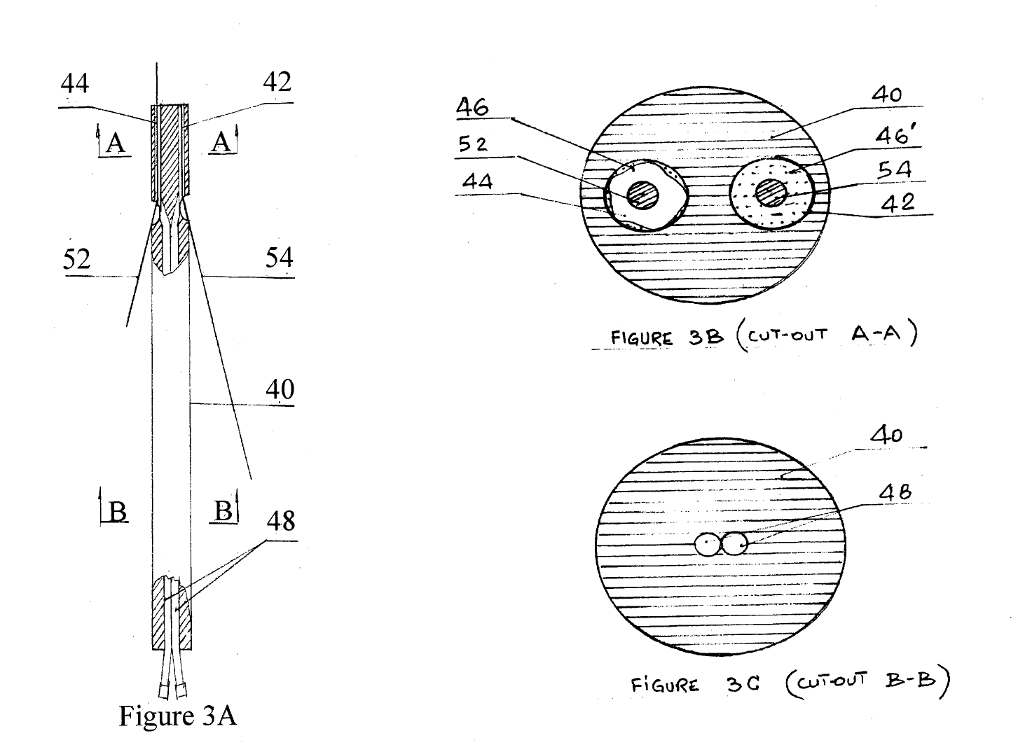

Operation of the device of this embodiment will now be shown schematically, with reference to FIGS. 2A, 2B, 2C, 2D and 2E, showing use of the device 10 of FIG. 1A during catheterization, by way of example only. First, a guidewire 30 is advanced through a coronary artery 32 until it is unable to move further, here because of the stenosis 34 (FIG. 2A). A new wire 36, which may be a replacement guidewire, is threaded through barrel 18 in wire advancement device (WAD) 10, and extends out of side port 20, as seen in FIG. 1A. New wire 36 is held in place in WAD 10 by the operator against the side of WAD 10, outside the body of the patient.

The end of original guidewire 30, extending from the patient, is threaded through the distal end of second barrel 16 in WAD 10, and extends through elongate barrel 16 and out of side port 20 of WAD 10. WAD 10 is advanced along guidewire 30 (FIG. 2B), providing wire-over-wire advancement through the artery 32. When WAD 10 reaches the tip of the original guidewire 30 in the coronary artery, the original guidewire 30, together with the WAD 10, are withdrawn over the new wire 36 (FIG. 2C), while wire 36 is held in place by the user (FIG. 2D). Now the new wire 36 is advanced beyond the stenosis 34 (FIG. 2E), permitting further treatment, such as advancing a balloon to the stenosis.

It will be appreciated that the method of the present invention can also be utilized in other applications, where it is desired to insert and advance a wire into a pipe system over a wire already in place.

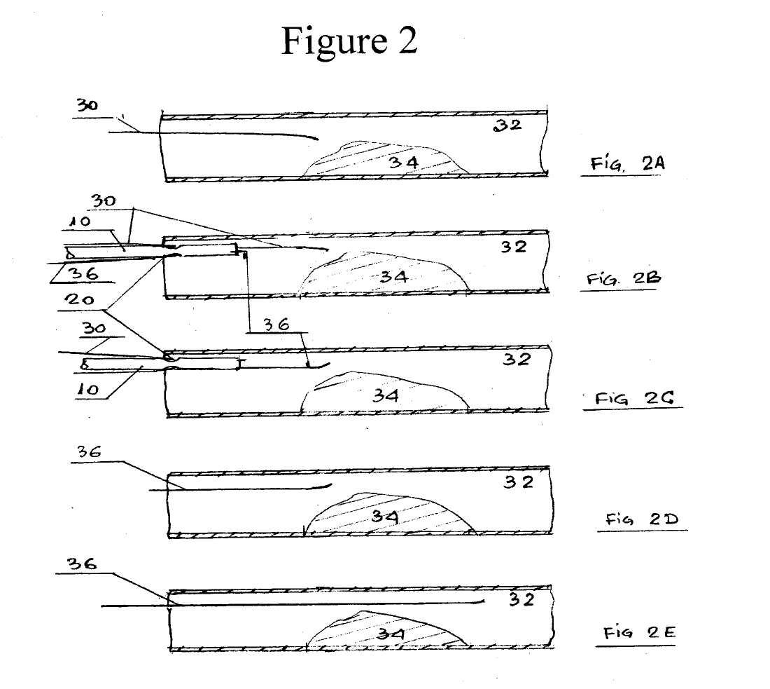

Referring now to FIG. 3A, there is shown a partially cut-away plan view of a device 40 for intravascular advancement of a wire constructed and operative in accordance with another embodiment of the present invention. WAD 40 is substantially similar to WAD 10 of FIG. 1A, and includes a flexible rod defining two barrels 42, 44 along at least a portion of its length.

Each of barrels 42 and 44 includes an inflatable annular balloon 46, 46′ in the interior of the barrel, lining the interior surface of the barrel, as seen most clearly in FIG. 3B. Balloons 46, 46′ are coupled, via inflation tubes 48 (FIGS. 3A and 3C), to a conventional screw-powered, handheld inflation device (not shown). In the deflated state (at 46 in FIG. 3B), the inflatable balloons leave a channel in the center of the barrel, for advancing along a wire 52, and, in an inflated state (at 46′ in FIG. 3B) substantially close the hollow barrel, so as to securely grip a wire 54 passing through the barrel. Differential inflation and deflation of each balloon will be accomplished by fluid injected under pressure by an inflation device via the inflation tubes 48.

Operation of the embodiment of FIG. 3a is substantially the same as that shown in FIGS. 2A, 2B, 2C and 2D, with reference to FIG. 1A. First, the guidewire 52 is advanced through a coronary artery until it is unable to move further, as because of a stenosis, or other difficulty in maneuvering the guidewire. The new wire 54, which may be a replacement guidewire, a special wire, or a medical carrier wire, for example, is threaded through barrel 42 of wire advancement device (WAD) 40. Balloon 46′ is inflated, via inflation tubes 48, to securely hold new wire 54.

The end of original guidewire 52, extending from the body of the patient, is threaded from the distal end through the second barrel 44 of WAD 40. The balloon 46 in barrel 44 remains deflated, so as to permit free advancement of WAD 40 over the original wire 52. When WAD 40 reaches the tip of the original guidewire 52, balloon 46 in barrel 44 will be inflated, in order to securely hold original guide wire 52, while balloon 46′ in barrel 42 is deflated, allowing replacement guidewire 54 to remain in place in the artery, while the original guidewire is withdrawn with the WAD, back over the replacement wire. Finally, the new wire 54 is advanced beyond the blockage, permitting further treatment.

There are several advantages of the suggested device over conventional methods of replacing a guidewire. The WAD will enable the operator to keep the original guidewire in the target vessel as far as it went, while advancing the new guidewire over it into the target vessel, and only then withdrawing the original guidewire. The procedure will conveniently be continued from a point where the new guidewire arrives at the most distant point reached by the original guidewire, taking advantage of the original guidewire position. In this way, the new wire is transferred over the original wire, without requiring the operator to manually guide the new wire until the point where the original wire was unable to continue, thereby reducing trauma to the patient, significantly reducing frustration in the operator, and shortening the length of the overall procedure. The WAD will also facilitate placement of special wires, such as laser wire, Doppler wire, pressure wire and distal protective devices (including occlusion devices and filters), which are less convenient to maneuver, compared to the conventional PCI guidewire. When two guidewires are required (for treating bifurcation lesions, the WAD will also facilitate placement of the second guidewire over the first one.

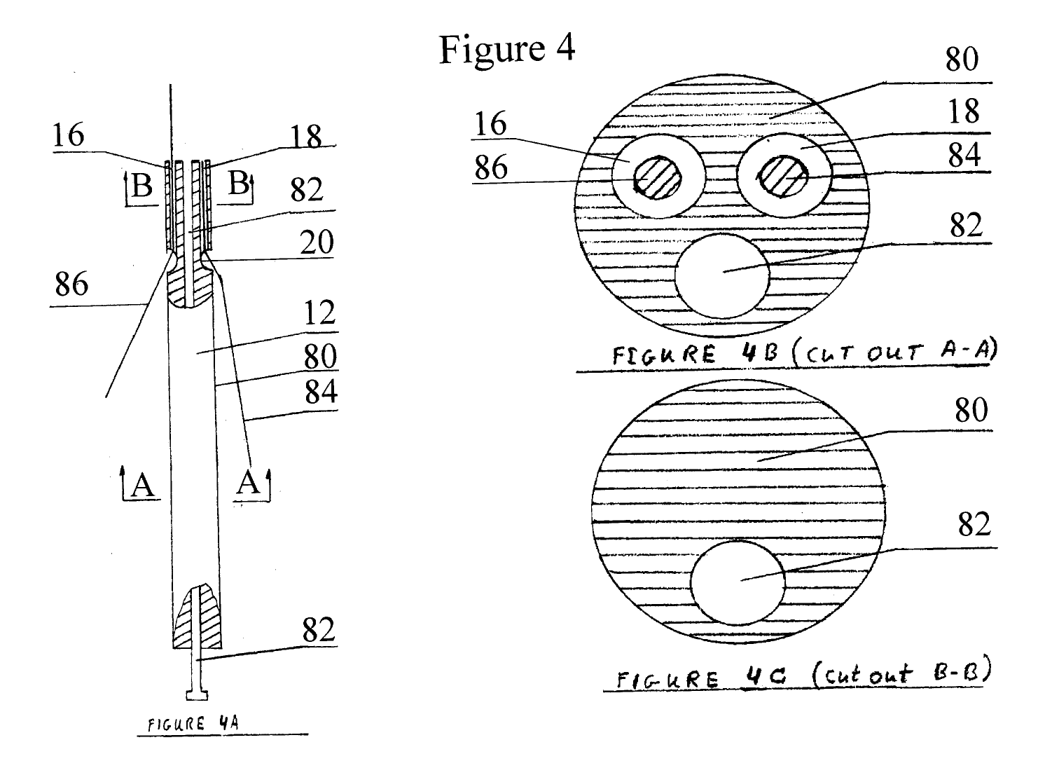

Referring now to FIG. 4a, there is shown a partially cut-away plan view of a wire advancement device (WAD) 80 for intravascular advancement of a wire 84, over a guide wire 86, according to an alternative embodiment of the present invention. FIGS. 4B and 4C are sectional illustrations through line A-A and B-B, respectively, in FIG. 4A. In this embodiment, the WAD 80 further includes a third barrel (or lumen) 82, extending through the solid WAD, from its distal end to its proximal end of the WAD. This barrel 82 is arranged for injection of a liquid through the WAD, from outside of a patient's body and into a vascular vessel. In particular, the system is useful for injecting a contrast material, such as dye, in order to allow verification of the wire position.

While the invention has been described hereinabove with respect to a limited number of embodiments, it will be appreciated that many variations, modifications, and other applications of the invention may be made. In particular, many variations on the method of holding the guidewire in the WAD can be utilized, on condition that the size remains suitable for intravascular use, when used in intravascular applications.

It will be appreciated that the invention is not limited to what has been described hereinabove merely by way of example. Rather, the invention is limited solely by the claims which follow.

1. A device for advancing a wire intravascularly, the device comprising:
a wire advancement device (WAD) adapted to hold a wire to be advanced, while the WAD is advancing wire-over-wire over a guidewire already in place.

2. A device for advancing a wire, the device comprising:
a flexible rod defining two barrels along at least a portion of its length;
one barrel arranged to hold and advance a wire,
the second barrel arranged for wire-over-wire advancement over a guidewire already in place.

3. The device according to claim 2, wherein said device includes:
a flexible rod defining two elongate barrels; and
an inflatable balloon lining an interior surface of each barrel,
said balloons, in an inflated state, being adapted to securely hold a wire and, in a deflated state, permitting free movement along a wire;
said balloons being coupled, via inflation tubes, to an inflation device.

4. The device according to claim 1, wherein said wire is a replacement guidewire.

5. The device according to claim 2, wherein said wire is a replacement guidewire.

6. The device according to claim 1, wherein said wire is selected from the group consisting of a laser wire, a Doppler wire, a medical carrier wire, a pressure wire and distal protective devices.

7. The device according to claim 1, and further comprising a third barrel, extending from a proximal end to a distal end of the WAD, said third barrel being arranged for injection of a liquid through the WAD

8. The device according to claim 2, and further comprising a third barrel, extending from a proximal end to a distal end of the WAD, said third barrel being arranged for injection of a liquid through the WAD

9. A method for advancing a wire, the method comprising:
holding the wire in a wire advancing device; and
advancing said wire advancing device by wire-over-wire advancement over a guidewire already in place.

10. The method according to claim 9, wherein said step of holding includes:
threading the wire through a first barrel in a flexible rod having two barrels,
said first barrel being arranged to hold said wire, and
threading an end of said guidewire through said second barrel, said second barrel being arranged for wire-over-wire advancement over said guidewire.

11. The method according to claim 10, wherein said step of threading includes threading said wire through a side port in said first barrel, and through and out a distal end of said first barrel.

12. The method according to claim 10, and comprising:
threading said wire through a first barrel of a wire advancement device (WAD), said first barrel being lined with a first inflatable balloon;
inflating said first balloon so as to hold said wire;
threading an outer end of said guidewire through a second barrel of said WAD, said second barrel being lined with a deflated second inflatable balloon; and
advancing said WAD over said guidewire.

13. The method according to claim 11, and comprising:
threading said wire through a first barrel of a wire advancement device (WAD), said first barrel being lined with a first inflatable balloon;
inflating said first balloon so as to hold said wire;
threading an outer end of said guidewire through a second barrel of said WAD, said second barrel being lined with a deflated second inflatable balloon; and
advancing said WAD over said guidewire.

14. The method according to claim 12, further comprising:
deflating said first inflated balloon, so as to release said wire;
inflating said second inflatable balloon so as to hold said guidewire; and
withdrawing said WAD and guidewire over said wire.

15. The method according to claim 14, further comprising:
deflating said first inflated balloon, so as to release said wire;
inflating said second inflatable balloon so as to hold said guidewire; and
withdrawing said WAD and guidewire over said wire.

16. The method according to claim 9, further comprising injecting a liquid through said WAD through a third barrel which extends from a proximal end to a distal end of the WAD.

17. The method according to claim 16, wherein said step of injecting includes injecting a contrast material through said third barrel to permit verification of the wire position.

18. The method according to claim 10, further comprising injecting a liquid through said WAD through a third barrel which extends from a proximal end to a distal end of the WAD.

19. The method according to claim 18, wherein said step of injecting includes injecting a contrast material through said third barrel to permit verification of the wire position.
